(19) United States
(12) Patent Application Publication
(10) Pub. No.: US 2006/0046268 A1
(43) Pub. Date: Mar. 2, 2006

(54) Diagnosis of anemia and optimizing treatment for heart repair

(75) Inventors: Dianna L. Devore, Windsor (AU); David Norman Haylock, Melbourne (AU)

Correspondence Address: BOZICEVIC, FIELD & FRANCIS LLP, 1900 UNIVERSITY AVENUE, SUITE 200, EAST PALO ALTO, CA, 94303, US

(73) Assignee: Australian Stem Cell Centre Ltd.

(21) Appl. No.: 10/928,611

(22) Filed: Aug. 26, 2004

(51) Int. Cl. G01N 33/53; G01N 33/567

(52) U.S. Cl. 435/7.1; 435/7.21

(57) ABSTRACT

A method of diagnosis of anemia is disclosed whereby blood is withdrawn from a patient and the level of hematopoietic stem cells (HSC) in the blood are determined by detecting a cell surface protein chosen from ACE⁺, CD34⁺, CD38, CD90⁺ (thyl) and Lin⁻. Patients with low levels of HSC are treated and in particular patients whose levels have been reduced by drugs such as ACE inhibitors are treated using an intermittent treatment regimine of the invention.

### FIELD OF THE INVENTION

The invention relates generally to the field of diagnosing and treating patients and particularly to diagnosing anemia by detecting hematopoietic stem cells (HSC) and treating patients by the injection of a formulation of autologous ACE⁺ cells.

### BACKGROUND OF THE INVENTION

Specific renin-angiotensin systems have been observed to be present within the cells of specific organ systems such as, for example, the kidney, heart, brain, and blood vessels. (See FIG. 1) This pathway has become a very important target for treating disease including hypertension, heart failure, and renal failure. Modulation of the renin-angiotensin system is central to the treatment regime for congestive heart failure (CHF) and end stage renal failure, as decrease in hypertension relieves the burden on the ailing organ.

In the renin-angiotensin system, the angiotensin converting enzyme (ACE) converts the octapeptide angitensin I to angiotensin II (AII) by cleavage of a dipeptide. Blocking ACE reduces the cleavage, in turn reducing peripheral vascular resistance. This action reduces the myocardial oxygen consumption, thereby improving cardiac output and moderating left ventricular and vascular hypertrophy. Angiotensin II receptor blockers (ARBs) block the binding of angiotensin II (AII) to the AT II or (AII) receptor. ACE inhibitors and ARBs are thus used clinically to decrease arterial pressure, ventricular afterload, blood volume and hence ventricular preload, as well as inhibit and reverse cardiac and vascular hypertrophy.

ACE inhibitors are a first line therapy for most patients with CHF due to left ventricle systolic dysfunction. Many ACE inhibitors have been well studied in clinical trials and have been shown to reduce the rate of death and hospitalizations in individuals with heart failure. As symptoms progress, additional therapy consisting of a diuretic, a beta-blocker, a vasodilator, and possibly digoxin may be indicated.

The ARBs are a newer class of drugs commonly used to treat hypertension. By blocking angiotensin II, ARBs help relax and dilate the blood vessels, lowering blood pressure and decreasing the heart's workload, two important goals of treating heart failure.

Even with the use of ACE inhibitors and ARBs, quality of life for CHF sufferers is poor and worse than some other chronic diseases such as diabetes and chronic lung disease [J McMurray, H J Dargie. Diagnosis and management of heart failure. British Medical Journal 1994 308: 321-8.]. CHF mortality ranges from 50% over 5 years in mild heart failure [P A McKee, W P Castelli, P A McNamara, W B Kannel. The natural history of congestive heart failure: the Framingham study. New England Journal of Medicine 1971 285: 1441-6.] to 60% per year in severe cases [CONCENSUS Trial Study Group. Effects of enalapril on mortality in severe congestive heart failure. New England Journal of Medicine 1987 316: 1429-35.]; these figures are higher than breast and prostate cancer death rates. The CHF problem will increase (the so-called heart failure epidemic) because of the impact of treatment on other forms of heart disease (for example thrombolysis) and the ageing population [JGF Cleland. Heart failure: the epidemic of the millennium. Hospital Update 1994 January; 9-10.].

More recently, clinical trials have been initiated looking at the regenerative capacity of autologous hematopoietic stem cells when introduced into damaged tissue of a failing organ. See [Burt R, et al., Hematopoietic stem cell transplantation for cardiac and peripheral vascular disease 2003; Rafii S, Lyden D Therapeutic stem and progenitor cell transplantation for organ vascularization and regeneration]. Recent studies have suggested that marrow and blood hematopoietic stem cells may contribute to nonhematopoietic tissue repair in multiple organ systems. In animal models and more recently in limited human trials, unpurified marrow mononuclear cells and/or subsets of adult hematopoietic stem cells have been reported to contribute to neoangiogenesis. Recent preclinical and pioneering clinical studies have shown that introduction of bone marrow-derived endothelial and hematopoietic progenitors can restore tissue vascularization after ischemic events in limbs, retina and myocardium.

### SUMMARY OF THE INVENTION

A method of diagnosis of anemia is disclosed whereby blood is withdrawn from a patient and the level of hematopoietic stem cells (HSC) in the blood are determined by detecting a cell surface protein chosen from ACE⁺, CD34⁺, CD38, CD90⁺ (thyl) and Lin⁻. Patients with low levels of HSC are treated and in particular patients whose levels have been reduced by drugs such as ACE inhibitors are treated using an intermittent treatment regimen of the invention.

A method of detection to optimize drug dosage is provided, whereby the number and/or activity of a patient's HSC cells are monitored to ensure administration of a therapeutically effective amount of a drug which blocks the renin-angiotensin system (RASBs), including combinations of RASBs, without undue decrease in the number and/or activity of hematopoietic stem cells (HSC) in the patient. The decrease in numbers or activity of the HSC may be determined by monitoring the patient's ACE⁺ HSC levels. The HSC levels may be monitored prior to the patient receiving the initial dosage of the RASB to set a baseline level of ACE⁺ HSC, and the levels monitored throughout the treatment regime to help set the appropriate levels of drug to maintain activity but prevent associated anemia.

In a specific embodiment, the monitoring is used to enhance efficacy of a therapeutic intervention to increase vascularization and regeneration of damaged tissue. The method comprises 1) ceasing administration of the renin-angiotensin system blocker (RASB) to allow recovery of ACE⁺ HSC; 2) monitoring the levels of HSC cells in a patient; and 3) administering ACE⁺ HSC to the patient. The HSC levels may be monitored by detecting levels of ACE⁺ cells in the patient's peripheral blood. The cells administered to the patient are preferably autologous cells isolated from the patient, but may also be allogeneic or derived from an alternate cell source, e.g., cells differentiated from embryonic stem cells. Following administration, the dosage of the RASB can optionally be continued. At that point the drug is again administered to the patient whereby the patient receives the benefit of ACE RASB without the adverse effect of the HSC levels being continually depleted below a desirable level. The RASB is preferably an ACE inhibitor, an ARB, or a combination thereof.

An aspect of the invention is a method of preventing anemia while treating congestive heart failure (CHF), comprising the steps of:
(a) administering to a patient a therapeutically effective amount of a pharmaceutically active drug, wherein such drug decreases the hematopoietic stem cells (HSC) in the patient;
(b) discontinuing the administering as in step (a) to allow recovery of the level of HSC while administering a formulation of HSC to the patient wherein the HSC may be autologous; and
(c) re-administering the drug to a patient as per step (a) following recovery of HSC levels in the patient.

Another aspect of the method of the invention, further comprises:
(d) monitoring the level of HSC following step (b) to determine recovery of HSC levels.

Another aspect of the invention, further comprises:
repeating the steps (a), (b) and (c) a plurality of times;
wherein the drug is a drug which blocks activation of the patient's renin-angiotensin systems (RASB). In a specific embodiment, the RASB is an Angiotensin Converting Enzyme (ACE) inhibitor. In another specific embodiment, the RASB is an Angiotensin II receptor antagonist.

In yet another aspect of the invention the drug is a combination of an Angiotensin Converting Enzyme (ACE) inhibitor and an Angiotensin II receptor antagonist.

In another aspect of the invention the monitoring step (d) is carried out by detecting levels of Angiotensin Converting Enzyme positive (ACE⁺) cells in the patient's peripheral blood.

In still another aspect of the invention the monitoring step is carried out by detecting levels of a cell surface protein in the patient's peripheral blood wherein the protein is chosen from CD34⁺, CD38; CD90⁺ (thyl), and Lin⁻.

Still yet another aspect of the invention further comprises:
repeating the steps (a), (b) and (c) a plurality of times;
wherein the pharmaceutically active drug is a combination of an Angiotensin Converting Enzyme (ACE) inhibitor and an Angiotensin II receptor antagonist; and
further wherein the administering (a) is carried out by administering drug on a daily basis or more frequently for three weeks or more and the discontinuing, and step (b) is carried out for three weeks or more.

Another aspect of the invention comprises a method of increasing vascularization and regeneration of damaged tissue, comprising the steps of:
(a) administering to a patient a therapeutically effective amount of a pharmaceutically active drug (or combination of drugs) which blocks activation of the patient's renin-angiotensin systems (RASB);
(b) determining a level of hematopoietic cells in the patient;
(c) discontinuing the administering as in step (a) when the level of hematopoietic cells as determined in step (b) drops below a given level;
(d) continuing the administering as in step (a) when the level of hematopoietic cells as determined in step (b) rises above a given level.

In another aspect of the invention the pharmaceutically active drug decreases the concentration of hematopoietic cells in the patient and the drug may be an Angiotensin Converting Enzyme (ACE) inhibitor such as Enalapril or Captropril alone or in combination with an Angiotensin II receptor antagonist such as bradykinin.

In another aspect of the invention the determining step (b) is carried out by detecting Angiotensin-converting Enzyme of hematopoietic cells.

In yet another aspect, the invention further comprises:
isolating HSC from a patient histocompatible with the patient; and
   introducing the isolated HSC to a patient histocompatible with the patient from which they were isolated. This includes HSC isolated from umbilical cord blood.

In still another aspect, the invention further comprises isolating HSC derived from an alternate source, e.g., differentiating HSC from embryonic stem cells. Such procedures are described in U.S. Pat. Nos. 6,613,568 and 6,280,715, which are incorporated by reference.

In still another aspect of the invention the isolated HSC are delivered to an organ of the patient (e.g. by injection) chosen from heart, kidney, liver and lung.

In another aspect of the invention the isolated HSC are administered to the same patient from which they are isolated.

Another aspect of the invention comprises a method of treating a patient, comprising the steps of:
monitoring hematopoietic stem cells (HSC) in a patient by determination of the patient's Angiotensin Converting Enzyme (ACE) level to obtain a monitored level; and
administering to the patient a combination of an ACE inhibitor and Angiotensin II receptor antagonist based on the monitored level.

In another aspect of the invention the monitoring the level of HSC comprises the steps of:
obtaining a cell sample including HSC or progeny thereof;
combining the sample with a labeled antibody which binds a protein on a HSC surface or a fragment thereof;
detecting the presence of the protein wherein the protein is chosen from CD34⁺, CD38⁻, CD90⁺, (thyl), and Lin⁻; and
identifying the HSC or progeny thereof having the protein or a fragment thereof by detecting the presence of the label on the antibody bound to the protein.

In another aspect of the invention the isolating of HSC comprises the steps of:
obtaining a cell population comprising HSC or progeny thereof;
detecting the presence of a protein or a fragment thereof on a cell of the population; and
selecting for cells which are identified by the presence of the protein or a fragment thereof on the cell.

In yet another aspect of the invention the isolating of HSC comprises the steps of:
obtaining cell populations comprising HSC or progeny thereof;
combining the cell population with a labeled antibody which binds a protein wherein the protein is chosen from CD34⁺, CD38⁻, CD90⁺, (thyl), and Lin⁻ or a fragment thereof expressed on a surface of an HSC; and
selecting for cells which are identified by the presence of the label on the antibody binding the protein or a fragment thereof on the HSC.

Another aspect of the invention comprises a method of treating anemia in a patient suffering from Coronary Heart Failure (CHF), comprising the steps of:
isolating angiotensin converting enzyme positive (ACE⁺) cells from a patient;
formulating the ACE⁺ cells into an injectable formulation; and
injecting a therapeutically effective amount of the formulation into the patient.

An aspect of the invention is a method of monitoring adverse side effects from a treatment regime by measuring levels of ACE⁺ cells in the peripheral blood of a patient receiving an RASB.

In another aspect of the invention is a new treatment protocol for the administration of angiotensin II receptor agonists.

Yet another aspect of the invention is a new treatment protocol for treating coronary heart failure patients with a combination of both ACE inhibitors and angiostatin II receptor angonists.

These and other aspects of the invention will become apparent to those skilled in the art upon reading this disclosure in combination with the figures attached hereto.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a schematic drawing showing the pathways by which ACE inhibitors work to block the cleavage of angiotensin I.

### DETAILED DESCRIPTION OF THE INVENTION

Before the present hematopoietic stem cell formulations and methods of treatment using such are described, it is to be understood that this invention is not limited to methods of treatment described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a stem cell” includes a plurality of cells, reference to “an ACE inhibitor” includes a plurality of such ACE inhibitors and reference to “the method of administering” includes reference to one or more methods of administering and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

ACE inhibitors are angiotensin converting enzyme inhibitors (e.g. Enalapril and Captropril). The angiotensin converting enzyme converts the octapeptide angitensin I to angiotensin II (AII) by cleavage of a dipeptide. An ACE inhibitor reduces the cleavage. These compounds can be administered in order to reduce peripheral vascular resistance via blockage of the angiotensin converting enzyme. This action reduces the myocardial oxygen consumption, thereby improving cardiac output and moderating left ventricular and vascular hypertrophy.

Angiotensin II receptor antagonists are compounds administered to block the binding of angiotensin II (AII) to the AT II or (AII) receptor, These compounds are referred to as AII receptor blockers (ARBs).

The terms “excipient material” “carrier and the like are intended to mean any compound forming a part of the formulation which is intended to act merely as a filler, carrier or the like i.e. not intended to have biological activity itself.

The terms “treating”, and “treatment” and the like are used herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a cell, group of cells or tissue, animal, mammal, and particularly a human, and includes: (a) preventing the disease from occurring in a subject which may be predisposed to the disease but has not yet been diagnosed as having it; (b) inhibiting the disease, i.e. arresting it's development; or (c) relieving the disease, i.e. causing regression of the disease and/or it's symptoms or conditions. The invention is directed towards treating patient's suffering from anemia while being treated for coronary heart failure (CHF). The present invention is involved in preventing, inhibiting, or relieving adverse effects attributed to CHF such as breathlessness, fatigue, and fluid retention resulting in an impaired ability of the heart to pump at normal levels of efficiency. The invention further comprises methods of treatment which result in increasing vascularization and/or regeneration of damaged tissue including heart, liver, kidney and lung tissue.

The terms “synergistic”, “synergistic effect” and the like are used interchangeably herein to describe improved treatment effects obtained by combining one or more different components of the invention. Although a synergistic effect in some fields means an effect which is more than additive (e.g., one plus one equals three) in the field of treating anemia, CHF and related diseases an additive (one plus one equals two) or less than additive (one plus one equals 1.2) effect may be synergistic. For example, if a patient has CHF and low levels of HSC, treatment to relieve the CHF with a combination of HSC and a biological matrix such as non-cellular submucosa which relieves a symptom of CHF more than either alone is a synergistic result. In connection with the present invention co-administration of HSC in a matrix along with co-administration of formulations of ACE inhibitors and angiotensin II receptor antagonists with the method of the invention whereby HSC are monitored and treatment intermittently administer based on HSC levels makes it possible to obtain improved effects which are synergistic, i.e. greater than the effects obtained by the administration of any of these components alone.

### Treating CHF with Drugs

The present invention is being described primarily for determination of regenerative capacity in treating patients with CHF. However, determining and monitoring can be used in the treatment of other chronic diseases such as chronic obstructive pulmonary disorder or end stage renal failure.

Certain ACE inhibitors have been shown to impact directly on the function of the hematopoietic system, in a manner that can be reversed upon ceasing administration of the drug. For example, the ACE inhibitor Enalapril was shown to cause reversible anemia in renal transplant patients. Vlahakos et al., Enalapril-associated amenia in renal transplant recipients treatedfor hypertension, Am J Kidney Dis.; 17:199-205 (1999). ARBs have also been shown to reversibly inhibit hematopoiesis in patients taking this class of drugs. The drug Losartan has been shown to reversibly decrease haemoglobin and hematocrit levels in patients with chronic obstructive pulmonary disease. Vlahakos, D V and Kosmas, E N. Losartan Reduces Hematocrit in Patients with Chronic Obstructive Pulmonary Disease and Secondary Erythrocytosis, Annals of Internal Medicine; 134:426-7 (2001).

The renin-angiotensin system plays an important role in regulating arterial pressure and both cardiac and vascular function. Renin release is generally in the kidney in response to renal artery hypotension and decreased sodium delivery to the distal tubules. Renin is an enzyme that acts upon a circulating substrate, angotensiongen, which undergoes proteolytic cleavage to form the angiotension I decapeptide. Angiotensin converting enzyme (ACE) converts the octapeptide angiotensin I to angiotensin II (All) by cleavage of dipeptide.

This pathway has become a very important target for treating hypertension and heart failure. ACE inhibitors and All receptor blockers (ARBs) are used clinically to decrease arterial pressure, ventricular afterload, blood volume and hence ventricular preload, as well as inhibit and reverse cardiac and vascular hypertrophy.

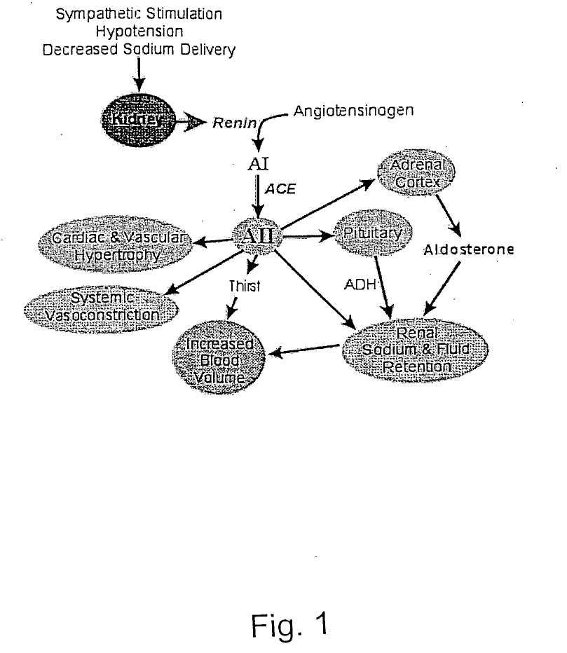

Angiotensin Converting Enzyme (ACE) inhibitors and Angiotensin II Receptor Blockers (ARBs) are vasodilators, which reduce the workload of the failing heart commonly seen with heart failure. As noted above, angiotensin II is created by the cleavage of angiotensin 1 by ACE, and thus these two classes of drugs work directly through the same pathway as shown in FIG. 1.

ACE inhibitors are a first line therapy for most patients with CHF due to left ventricle systolic dysfunction. Many ACE inhibitors have been well studied in clinical trials and have been shown to reduce the rate of death and hospitalizations in individuals with heart failure. As symptoms progress, additional therapy consisting of a diuretic, a beta-blocker, a vasodilator, and possibly digoxin may be indicated.

The ARBs are a newer class of drugs commonly used to treat hypertension. By blocking angiotensin II, ARBs help relax and dilate the blood vessels, lowering blood pressure and decreasing the heart's workload, two important goals of treating heart failure.

Both ACE inhibitors and ARBs have been shown to suppress hematopoiesis in different patient populations.

ACE is believed to enhance the recruitment of early hematopoietic progenitor cells into S-phase in the bone marrow. ACE inhibitors have been shown to reduce the circulating hematopoietic progenitors in healthy subjects. Certain ACE inhibitors have been shown to impact directly on the function of the hematopoietic system, in a manner that can be reversed upon ceasing administration of the drug. For example, the ACE inhibitor Enalapril was shown to cause reversible anemia in renal transplant patients. Vlahakos et al., Enalapril-associated amenia in renal transplant recipients treated for hypertension, Am J Kidney Dis.; 17:199-205 (1999). The present invention provides a solution to the problem that ACE inhibitors, as well as being vasodilators, inhibit production of blood cells, and act as direct inhibitors of hematopoiesis.

ARBs have also been shown to reversibly inhibit hematopoiesis in patients taking this class of drugs. The drug Losartan has been shown to reversibly decrease haemoglobin and hematocrit levels in patients with chronic obstructive pulmonary disease. Vlahakos, D V and Kosmas, E N. Losartan Reduces Hematocrit in Patients with Chronic Obstructive Pulmonary Disease and Secondary Erythrocytosis, Annals of Internal Medicine; 134:426-7 (2001). It is not clear whether the effect is due to action solely within the same pathways as ACE, or whether there is redundancy in the pathways involved in this inhibitory activity.

While not committing to any particular mechanism of action it is proposed that a local renin-angiotensin system (RAS) is active in the bone marrow, and that this plays a role in regulating hematopoiesis. The RAS is thought to be an important determinate for erythropoiesis. RAS inactivation may confer susceptibility to hematocrit lowering effects of ACE inhibitors and ARBs. It is not clear whether it will also impact upon the vascularization ability of hematopoietic stem cells.

The present invention may be used for patients taking any ACE inhibitors, including the following exemplary drugs or a combination thereof: lisinopril (Zestril™), ramipril (Tritace™), trandolapril (Odrik™), captopril (Capoten™), fosinopril (Monopril™), enalapril (Renitec™), benazepril (Lotensin™), quinapril (Accupril™), and perindopril. (Coversyl™). The invention may also be used for patients taking any ARB, including the following exemplary drugs or a combination thereof: candesartan (Atacand™), telmisartan (Micardis™), irbesartan (Avapro™), losartan (Cozaar™), valsartan (Diovan™), olmesartan (Benicar™), and eprosartan (Teveten™).

### The Invention in General

Patients are monitored to determine the level of hematopoietic stem cells in the blood. Hematopoeitic stem cells (HSC) are known to express ACE on their surface (see PCT published application WO 98/23773 published Jun. 4, 1998) and a local renin-angiotensin system is involved in the microenvironment of the hematopoietic stem cell niche. It is particularly important to monitor levels of HSC in patients being treated with drugs which lower levels of HSC. For example, patients treated with either or both of an Angiotensin Converting Enzyme (ACE) inhibitor (e.g. Enalapril) and an Angiotensin II receptor antagonist (e.g. bradykinin) are often found to have lowered levels of HSC compared to their levels when treatment began.

The ability of a patient's hematopoietic system to aid in the regeneration of cardiac function is dependent in part on the ability of their hematopoietic stem cells to undergo cell division and differentiation. An ACE inhibitor blocks the ability of the hematopoietic stem cells to enter into S-phase, in effect blocking the ability of these cells to divide and produce other cell types necessary for the regeneration.

In connection with the present invention it has been recognized that with CHF patients undergoing ACE-inhibitor therapy, the benefits of the therapy in terms of control of hypertension and relief of load on the heart is offset by loss in regenerative capability and reduced levels of HSCs. However, the adverse effects of ACE inhibitors are reversible. Thus, in accordance with the present invention the hematopoietic capacity can be monitored both with and without the drug i.e. monitored over periods of time when one or more drugs are administered (treatment period) and periods of time when the drug is not administered (recovery periods). Identification of ACE⁺ cell levels in mobilized hematopoietic stem cells makes it possible to identify how long each period should be. The regenerative therapy may be merely temporarily stopping the administration of ACE inhibitor therapy and/or ARBs i.e. providing for a recovery period where no drug is administered. However, the recovery period may include injection of isolated hematopoietic stem cells into the circulatory system or directly into the patient's heart. Thus, identification of ACE levels, or levels of ACE activity, can be used as a monitoring methodology to determine the best time to treat a patient.

The identification of ACE⁺ cells may also be a surrogate marker for patients undergoing treatment with ARBs, as the blockage of the ARBs (particularly when combined with the administration of an ACE inhibitor) can lead to a decrease in the number of hematopoietic progenitor cells for use in regenerative therapy. Since ACE is a marker for such cells, identification of ACE⁺ cells can also be used to monitor the levels of hematopoiesis in patients undergoing ARB and/or ACE inhibitors therapy, and can be used to make treatment decisions such as those discussed.

Thus, identification of ACE⁺ cells is used in accordance with the present invention to aid directly in therapeutic decisions, such as 1) the decision to cease treatment with ACE inhibitors and/or ARB's for a period prior to regenerative intervention; 2) monitoring the level of ACE⁺ cells in such patients to identify recovery of ACE⁺ cell levels, and appropriate timing for introduction of HSC to the patient; and 3) identification of “responders”, i.e. people who will respond to therapeutic intervention versus patients without the appropriate regenerative capacity.

Anaemia from a variety of causes is relatively common in older patients who are the majority of recipients of ACE-inhibitor therapy and the exact cause is not always clear. Thus, identification of ACE⁺ cells is also used in connection with the present invention to confirm or exclude such therapy as the cause of the anaemia.

The present invention is being described primarily for determination of regenerative capacity in treating patients with CHF. However, determining and monitoring can be used in the treatment of other chronic diseases such as chronic obstructive pulmonary disorder or end stage renal failure.

The intermittent treatment method of the invention makes it possible to obtain the benefit of drug treatment while reducing the adverse side effects of the drugs. The method may be carried out by the daily administration of either or both of an ACE inhibitor and an ARB during a treatment period. Thereafter, either one or both the ACE inhibitor and ARB drug are not administered during a recovery period. Both the treatment period and recovery period last a plurality of days, weeks or months. During the recovery period the patient may be treated with the administration of a formulation of HSCs. Thus, in accordance with an example, the method of the invention can be carried out as follows:

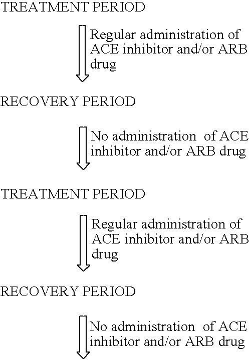

Both the treatment period and the recovery period can be for 1, 2, or 3 weeks, 1, 2, or 3 months and may be repeated many times over the life of the patient. Regular administration during this treatment period may be a dosage of the drug once daily, twice daily, once every other day, or as would be prescribed for regular use by a clinician skilled with dosage of the drug for treatment of the specific failing organ. The periods can be the same or different from each other in length. The method can be modified to include a step of monitoring levels of HSCs in the patient as follows:

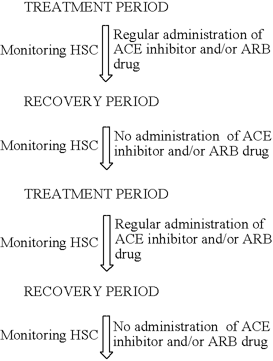

The monitoring step is carried out during the treatment period to detect when the patient's HSC level falls below a given desired point, e.g., the HSC levels have decreased a given percentage from pretreatment levels. When that level is reached the recovery period is started, and the regular dosage of the drug ceases for this period. Monitoring during the recovery period is carried out to determine the point when the patient's HSC level rises back to normal or to an acceptable point. When that point is reached the treatment period begins again. This intermittent treatment can be carried out a plurality of times and may be carried out for the patient's entire life if needed.

The method can be carried out on a large number of patients in clinical trials with monitoring. The monitoring data can be used statistically to determine the best length for both the treatment period and the recovery period. The periods may vary based on the age, condition, sex, weight and/or other parameters such the patient's HSC levels prior to any treatment.

This intermittent treatment methodology of the invention may be modified to be an alternating two types of treatment. This is carried out by adding the administration of a formulation of HSCs during the recovery period as follows:

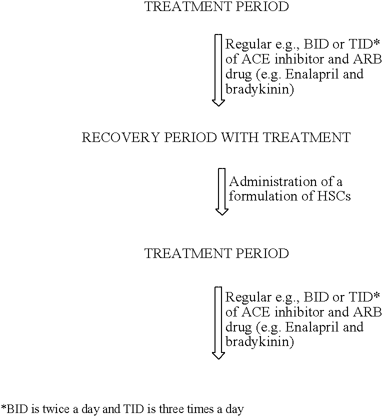

The recovery period may be shortened considerably by administering a HSC formulation as compared to a recovery period when no HSC formulation is administered.

Although the present invention is described in terms of treatments of heart failure, it would be obvious to one skilled in the art upon reading the present disclosure that the methods of the invention could be used for enhancing organ regeneration in any patient receiving an ACE inhibitor or an ARB. This includes patients with renal failure, chronic obstructive pulmonary discorder, liver failure and the like.

### Identifying Hematopoietic Progenitor Cells

The marker ACE along with the marker CD34 have the ability to identify and isolate an early hematopoietic progenitor cell. ACE binding agents may be used to isolate hematopoietic progenitors. As ACE can be used to preferentially isolate the hematopoietic progenitor populations with increased regenerative capacity, it can be used in connection with the present invention for identifying hematopoietic progenitors for many purposes, including the traditional use in bone marrow transplantation. This may increase the efficiency of transplantation by identifying a preferred transplantation cell population.

It is possible to detect HSC via methods disclosed in published PCT application WO 03/038071 published May 8, 2003 as well as the various publications cited therein. In this method a sample comprising HSC or progeny thereof is obtained. In the sample the presence of at least one carbohydrate sequence comprising a sequence of at least one disaccharide repeat of glucosome acid and N-acetylglucosamine or an equivalent thereof and identifying a HSC with that sequence. The HSC can be isolated as described in the WO 03/038071 application and publications cited therein all of which are incorporated herein.

The recombinant antibody can be produced by any recombinant means known in the art. Such recombinant antibodies include, but are not limited to, fragments produced in bacteria and non-human antibodies in which the majority of the constant regions have been replaced by human antibody constant regions. In addition, such “humanized” antibodies can be obtained by host vertebrates genetically engineered to express the recombinant antibody.

In addition, the monospecific domains can be attached by any method known in the art to another suitable molecule compound. The attachment can be, for instance, chemical or by genetic engineering.

The antibodies can be conjugated to other suitable molecules and compounds including, but not limited to, enzymes, magnetic beads, colloidal magnetic beads, haptens, fluorochromes, metal compounds, radioactive compounds, chromatography resins, solid supports or drugs. The enzymes that can be conjugated to the antibodies include, but are not limited to, alkaline phosphatase, peroxidase, urease and β-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P. Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m, ¹²⁵I and amino acids comprising any radionuclides, including, but not limited to ¹⁴C, ³H and ³⁵S.

Various techniques can be employed to separate or enrich the cells by initially removing cells of dedicated lineage. Monoclonal antibodies and binding proteins are particularly useful for identifying cell lineages and/or stages of differentiation. The antibodies can be attached to a solid support to allow for crude separation. The separation techniques employed should maximize the retention of viability of the fraction to be collected. Various techniques of different efficacy can be employed to obtain “relatively crude” separations. The particular technique employed will depend upon efficiency of separation, associated cytotoxicity, ease and speed of performance, and necessity for sophisticated equipment and/or technical skill.

Procedures for separation or enrichment can include, but are not limited to, magnetic separation, using antibody-coated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation or any other convenient technique.

The use of separation or enrichment techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye, Hoescht 33342).

Techniques providing accurate separation include, but are not limited to, FACS, which can have varying degrees of sophistication, e.g., a plurality of color channels, low angle and obtuse light scattering detecting channels, impedence channels, etc. Any method which can isolate and distinguish these cells according to levels of expression of cell surface markers may be used.

While it is believed that the particular order of separation is not critical to this invention, the order indicated is preferred. Preferably, cells are initially separated by a coarse separation, followed by a fine separation, with positive selection with antibodies to ACE or fragments thereof. It is one embodiment that a pre-enrichment step is applied which enriches CD34⁺ cells prior to isolation utilizing ACE.

To further enrich for any cell population, specific markers for those cell populations may be used. For instance, specific markers for specific cell lineages such as lymphoid, myeloid or erythroid lineages may be used to enrich for or against these cells. These markers may be used to enrich for HSCs or progeny thereof by removing or selecting out mesenchymal or keratinocyte stem cells.

The methods described above can include further enrichment steps for cells by positive selection for other stem cell specific markers. Suitable positive stem cell markers include, but are not limited to, CD34⁺, Thy-1⁺, and c-kit⁺. By appropriate selection with particular factors and the development of bioassays which allow for self-regeneration of HSCs or progeny thereof and screening of the HSCs or progeny thereof as to their markers, a composition enriched for viable HSCs or progeny thereof can be produced for a variety of purposes.

### Effects of Hematopoietic Stem Cells

The ability of a patient's hematopoietic system to aid in the regeneration of cardiac function is dependent in part on the ability of their hematopoietic stem cells to undergo cell division and differentiation. An ACE inhibitor, in blocking the ability of the hematopoietic stem cells to enter into S-phase, in effect blocks the ability of these cells to divide and produce other cell types necessary for the regeneration.

In connection with the present invention it has been recognized that with CHF patients undergoing ACE-inhibitor therapy, the benefits of the therapy in terms of control of hypertension and relief of load on the heart may be offset by loss in regenerative capability. However, the effects of ACE inhibitors are reversible. Thus, in accordance with the present invention the hematopoietic capacity can be monitored both with and without the drug. Identification of ACE+cell levels in mobilized hematopoietic stem cells makes it possible to identify the most opportune time for introduction of a regenerative therapy. The regenerative therapy may be merely temporarily stopping the administration of ACE inhibitor therapy and/or ARBs. However, it may include injection of isolated hematopoietic stem cells into the circulatory system or directly into the patient's heart. Thus, identification of ACE levels, or levels of ACE activity, can be used as a monitoring methodology to determine the best time to treat a patient.

The identification of ACE⁺ cells may also be a surrogate market for patients undergoing treatment with ARBs, as the blockage of the ARBs could lead to a decrease in the number of hematopoietic progenitor cells for use in regenerative therapy. Since ACE is a marker for such cells, identification of ACE⁺ cells can also be used to monitor the levels of hematopoiesis in patients undergoing ARB therapy, and can be used to make treatment decisions such as those discussed.

Thus, identification of ACE⁺ cells is used in accordance with the present invention to aid directly in therapeutic decisions, such as 1) the decision to cease treatment with ACE inhibitors for a period prior to regenerative intervention; 2) monitoring the level of ACE⁺ cells in such patients to identify recovery of ACE⁺ cell levels, and appropriate timing for introduction of HSC to the patient; and 3) identification of “responders”, i.e. people who will respond to therapeutic intervention versus patients without the appropriate regenerative capacity.

Anaemia from a variety of causes is relatively common in older patients who are the majority of recipients of ACE-inhibitor therapy and the exact cause is not always clear. Thus, identification of ACE⁺ cells is also used in connection with the present invention to confirm or exclude such therapy as the cause of the anaemia.

The present invention is being described primarily for determination of regenerative capacity in treating patients with CHF. However, determining and monitoring can be used in the treatment of other chronic diseases such as chronic obstructive pulmonary disorder or end stage renal failure.

### Injecting Isolated ACE⁺ Cells

In accordance with the methodology such as that described above it is possible to isolate ACE⁺ cells and concentrate those cells into a formulation. The concentrated cells can be formulated into an injectable formulation and then injected into a patient. For example, the cells can be injected into damaged myocardium. Preferably, the cells are extracted from a patient, concentrated, formulated, and injected back into the same patient so that the cells are autologous.

In accordance with one aspect of the invention the cells are formulated in a matrix. By dispersing the ACE⁺ cells within the submucosal material and then injecting the material into the patient such as the patient's heart, muscle, or other tissue material improved treatment effects can be obtained. The matrix may be a biological material which may be a non-cellular material which aids in tissue regeneration. Non-cellular matrix compositions are disclosed in a number of patents such as U.S. Pat. Nos. 4,902,508; 4,956,178; 6,696,270; and 6,653,291 as well as numerous patents and publications cited in these patents. An acellular tissue matrix that has been used successfully in a number of applications is the matrix prepared by the methods disclosed in U.S. Publ. Nos. 2003/0035843 and 2003/0143207 to S. A. Livesey, et al., and are incorporated by reference herein in their entirety. The methods involve processing biological tissue with a stabilizing solution to reduce damage to the tissue, decellularizing the tissue, treating the decellularized tissue with a cryoprotectant solution, then freezing and drying the tissue. The processed acellular tissue may then be stored and, ultimately, rehydrated for use. The conditions employed in these methods minimize damage to the functionality of the tissue.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of diagnosing anemia, comprising the steps of:
isolating blood from a patient;
detecting hematopoietic stem cells (HSC) in the blood and determining a concentration of HSC per unit volume of blood wherein the HSC are detected by detecting a cell surface protein on the HSC.

2. The method of claim 1, wherein the cell surface protein is Angiotensin Converting Enzyme (ACE).

3. The method of claim 1, wherein the cell surface protein is chosen from CD34⁺, CD38, CD90⁺ (thyl) and Lin⁻.

4. The method of claim 1, wherein the cell surface protein is detected with an antibody immunospecific for the protein.

5. The method of claim 4, wherein a plurality of different antibodies are used to detect a plurality of different cell surface proteins.

6. A method of treating anemia, comprising the steps of:
isolating angiotensin converting enzyme positive (ACE⁺) cells;
formulating the ACE⁺ cells into an injectable formulation; and
injecting a therapeutically effective amount of the formulation into a patient.

7. The method of claim 6, wherein the ACE⁺ cells are isolated from the blood of a patient by a cell surface protein on the ACE⁺ cells.

8. The method of claim 7, wherein the cell surface protein is chosen from CD34⁺, CD38, CD90⁺ (thyl) and Lin⁻.

9. The method of claim 7, wherein the cell surface protein is ACE.

10. The method of treating congestive heart failure comprising the steps of:
(a) administering to a patient a therapeutically effective amount of a pharmaceutically active drug which decreases arterial pressure, ventricular afterload, blood volume and ventricular preload, wherein such drug decreases the number of hematopoietic stem cells (HSC) in the patient;
(b) discontinuing the administering as in step (a) to allow recovery of the level of HSC;
(c) administering the drug to a patient as per step (a) following recovery of HSC levels in the patient; and
(d) monitoring the level of HSC following step (b) to determine recovery of HSC levels.

11. The method of claim 10, further comprising:
injecting a formulation of autologous ACE⁺ cells into the patient's heart.

12. The method of claim 11, wherein the pharmaceutically active drug further comprises an angiotensin II receptor antagonist.

13. The method of claim 10, wherein the monitoring step (d) is carried out by detecting levels of Angiotensin Converting Enzyme positive (ACE⁺) cells in the patient's peripheral blood.

14. The method of claim 10, wherein monitoring step (d) is carried out by detecting levels of a cell surface protein selected from CD34⁺, CD38, CD90⁺, (thyl) and Lin⁻.

15. The method of claim 10, wherein the pharmaceutically active drug is an Angiotensin Converting Enzyme (ACE) inhibitor, an Angiotensin II receptor antagonist, and/or a combination thereof.

16. A method of treating a patient, comprising the steps of:
diagnosing the patient as having congestive heart failure;
monitoring hematopoietic stem cells (HSC) in a patient by determination of the patient's Angiotensin Converting Enzyme (ACE) positive blood cells to obtain a base level; and
administering a therapeutic amount of an ACE inhibitor.

17. The method of claim 16, wherein the monitoring the level of HSC comprises the steps of:
obtaining a cell sample including HSC or progeny thereof;
detecting the presence of the cell surface protein associated with HSC; and
identifying the HSC or progeny thereof having the cell surface protein or a fragment thereof by detecting the presence of the binding protein on the HSC or progeny thereof.

18. The method of claim 16, wherein the HSC are obtained by a process which comprises the steps of:
obtaining a cell population comprising HSC or progeny thereof;
detecting the presence of a cell surface protein associated with HSC; and
selecting for cells which are identified by the presence of the cell surface protein or a fragment thereof on the HSC.

19. The method of claim 16, further comprising:
further monitoring hematopoietic stem cells (HSC) in a patient; and
adjusting the therapeutic amount of the ACE inhibitor based on the HSC levels.

20. A method of treating a patient, comprising the steps of:
diagnosing the patient as having congestive heart failure;
monitoring hematopoietic stem cells (HSC) in a patient by determination of the patient's Angiotensin Converting Enzyme (ACE) positive blood cells to obtain a base level; and
administering a therapeutic amount of an Angiotensin II receptor antagonist.

21. The method of claim 20, wherein the monitoring the level of HSC comprises the steps of:
obtaining a cell sample including HSC or progeny thereof;
detecting the presence of the cell surface protein associated with HSC; and
identifying the HSC or progeny thereof having the cell surface protein or a fragment thereof by detecting the presence of the binding protein on the HSC or progeny thereof.

22. The method of claim 20, wherein the HSC are obtained by a process which comprises the steps of:
obtaining a cell population comprising HSC or progeny thereof;
detecting the presence of a cell surface protein associated with HSC; and
selecting for cells which are identified by the presence of the cell surface protein or a fragment thereof on the HSC.

23. The method of claim 20, further comprising:
further monitoring hematopoietic stem cells (HSC) in a patient; and
adjusting the therapeutic amount of the Angiotensin II receptor antagonist based on the HSC levels.
